(19) United States
(12) Patent Application Publication
(10) Pub. No.: US 2024/0228526 A1
(43) Pub. Date: Jul. 11, 2024

(54) METHOD FOR PREPARING ANTI-HIV DRUG ISLATRAVIR

(71) Applicant: FUJIAN RIBIO TECHNOLOGY CO., LTD., Fujian (CN)

(72) Inventors: Yibiao LIU, Fujian (CN); Yazhou LI, Fujian (CN); Zhenchang CHEN, Fujian (CN); Hongjuan ZHANG, Fujian (CN)

(21) Appl. No.: 18/560,414

(22) PCT Filed: Oct. 11, 2022
(86) PCT No.: PCT/CN2022/124483

§ 371 Date: Nov. 13, 2023

(30) Foreign Application Priority Data
Dec. 13, 2021 (CN) 202111515780.X

(51) Int. Cl. C07H 19/173

(52) U.S. Cl. CPC C07H 19/173

(57) ABSTRACT

Provided is a method for preparing islatravir, including: 1) subjecting compound of Formula II as a raw material to selective hydroxyl protection reaction to obtain compound of Formula III, 2) subjecting the compound of Formula III to hydroxyl oxidation reaction to obtain compound of Formula IV; 3) subjecting the compound of Formula IV and dimethyl diazomethylphosphonate to reaction under an alkaline condition to obtain compound of Formula V; 4) subjecting the compound of Formula V to protecting group conversion reaction to obtain compound of Formula VI; 5) subjecting the compound of Formula VI to glycosylation reaction to obtain compound of Formula VII; 6) subjecting the compound of Formula VII to selective deprotection reaction to obtain compound of Formula VIII; 7) subjecting the compound of Formula VIII to deoxidation reaction to obtain compound of Formula IX; and 8) subjecting the compound of Formula IX to debenzylation reaction to obtain the islatravir.

### CROSS REFERENCE TO RELATED APPLICATION

The application claims the benefit and priority of the Chinese Patent Application No. CN202111515780X entitled “METHOD FOR PREPARING ANTI-HIV DRUG ISLATRAVIR”, filed with the China National Intellectual Property Administration (CNIPA) on Dec. 13, 2021, which is incorporated herein by reference in its entirety as part of the application.

### TECHNICAL FIELD

The present disclosure relates to the technical field of chemical synthesis, and in particular to a method for preparing an anti-HIV drug islatravir.

### BACKGROUND

Islatravir (formerly known as EFdA or MK-8591) is an innovative nucleoside reverse transcriptase inhibitor (NRTI) for the treatment of HIV infection with multiple mechanisms of action. In addition to acting as a defective component that prevents the construction of new DNA strands, the islatravir may also play a role in subsequent steps during the viral replication. Due to an ultra-long half-life, islatravir has a potential to be used in HIV treatment with fewer administrations and even in pre-exposure prophylaxis (PrEP). Clinical studies have shown that oral administration or subcutaneous implantation of the islatravir has outstanding safety and tolerability, showing desirable application prospects. The islatravir has a chemical structure shown in Formula I:

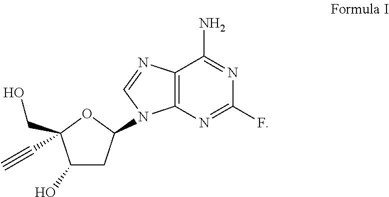 [SMILES: C#C[C@]1(CO)O[C@@H](n2cnc3c(N)nc(F)nc32)C[C@@H]1O]

Islatravir is a new type of nucleoside analog, and many research teams have conducted in-depth studies on islatravir synthesis. Kuwaraha's team has proposed two total synthetic routes for islatravir in 2011 (Org. Lett., 2011, 13, 5264-5266) and 2015 (Org. Lett., 2015, 17, 828-831), respectively, one using (R)-propylidene glyceraldehyde as a raw material and undergoing 12 steps, with a comprehensive yield of 18%, and the other using diacetone glucose as a raw material and undergoing 15 steps, with a comprehensive yield of 37%. McLaughlin et al. have proposed in 2017 (Org. Lett., 2017, 19, 926-929) that the islatravir could be synthesized by using glycerone diacetate as a raw material through a 15-step reaction, with a comprehensive yield of 15%. The above-mentioned synthetic routes have long processes and low yields, and are not easy to be industrialized. Whittaker's team has proposed in 2020 (Chemistry—A European Journal, 2020, 26, 14118-14123) that the islatravir could be synthesized by glycosylation reaction through biocatalysis; after designing and trying a series of substrates, it was decided that a key intermediate was prepared by using p-methoxybenzaldehyde or p-butyrylanisole as a raw material and selecting a suitable ligand, and then subjecting them to 4-step reaction; and the islatravir was then synthesized from the key intermediate by an enzymatic method, with a comprehensive yield of 47%. The raw materials and ligands required for this route are difficult to purchase, making industrialization difficult.

### SUMMARY

In view of the deficiencies in the existing methods for preparing islatravir, the present disclosure aims to provide a method for preparing islatravir which has a relatively short route, exhibits a high yield, and is suitable for industrialization.

To achieve the above object, the present disclosure provides a method for preparing islatravir by subjecting 3-O-benzyl-4-C-hydroxymethyl-1,2-O-isopropylidene-α-D-ribofuranose (a compound of Formula II) as a raw material to 8 steps reactions in sequence according to the following steps.

The method for preparing islatravir includes the following steps:
1) subjecting a compound of Formula II as a raw material to selective hydroxyl protection reaction to obtain a compound of Formula III,

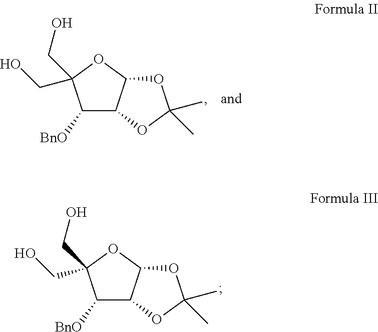

2) subjecting the compound of Formula III to hydroxyl oxidation reaction to obtain a compound of Formula IV,

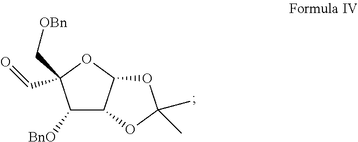 [SMILES: CC1(C)O[C@H]2O[C@](C=O)(COCc3ccccc3)[C@@H](OCc3ccccc3)[C@H]2O1]

3) subjecting the compound of Formula IV and dimethyl diazomethylphosphonate to reaction under an alkaline condition to obtain a compound of Formula V, such that a terminal alkynyl group of the islatravir is constructed,

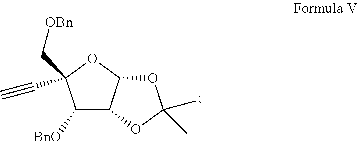 [SMILES: C#C[C@]1(COCc2ccccc2)O[C@@H]2OC(C)(C)O[C@@H]2[C@@H]1OCc1ccccc1]

4) subjecting the compound of Formula V to protecting group conversion reaction, such that protection by one propylidene group is converted to protection by two acetyl groups to obtain a compound of Formula VI,

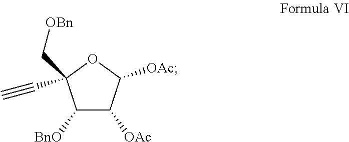 [SMILES: C#C[C@]1(COCc2ccccc2)O[C@H](OC(C)=O)[C@H](OC(C)=O)[C@@H]1OCc1ccccc1]

5) subjecting the compound of Formula VI to glycosylation reaction, such that a glycosidic bond is constructed to obtain a compound of Formula VII,

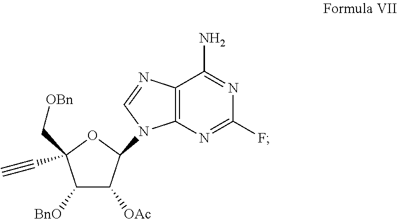 [SMILES: C#C[C@]1(COCc2ccccc2)O[C@@H](n2cnc3c(N)nc(F)nc32)[C@H](OC(C)=O)[C@@H]1OCc1ccccc1]

6) subjecting the compound of Formula VII to selective deprotection reaction to remove the acetyl groups to obtain a compound of Formula VIII,

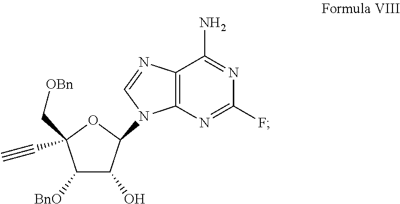 [SMILES: C#C[C@]1(COCc2ccccc2)O[C@@H](n2cnc3c(N)nc(F)nc32)[C@H](O)[C@@H]1OCc1ccccc1]

7) subjecting the compound of Formula VIII to deoxidation reaction to remove a hydroxyl group at a 2′-position to obtain a compound of Formula IX,

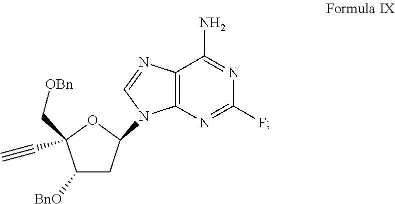 [SMILES: C#C[C@]1(COCc2ccccc2)O[C@@H](n2cnc3c(N)nc(F)nc32)C[C@@H]1OCc1ccccc1]

and
8) subjecting the compound of Formula IX to debenzylation reaction to remove two benzyl groups to produce the islatravir.

In some embodiments, in step 1), the selective hydroxyl protection reaction is conducted under the action of sodium hydride and benzyl bromide by using N,N-dimethylformamide (DMF) as a solvent.

In some embodiments, the selective hydroxyl protection reaction is conducted at a temperature of −10° C. to 40° C., specifically −10° C., 0° C., 20° C., 10° C. to 40° C., or −5° C. to 20° C.; and the selective hydroxyl protection reaction is conducted for 6 h to 24 h, specifically 8 h, 12 h, 16 h, or 24 h.

In some embodiments, in the selective hydroxyl protection reaction, a molar ratio of the compound of Formula II, the sodium hydride, and the benzyl bromide is in a range of 1:(0.8-1.2):(0.8-1.2), specifically 1:(0.8-1.0):(0.8-1.0) or 1:(1.0-1.2):(1.0-1.2).

In some embodiments, in step 2), the hydroxyl oxidation reaction is conducted under the action of a Dess-Martin oxidant by using dichloromethane (DCM) as a solvent.

In some embodiments, the hydroxyl oxidation reaction is conducted at a temperature of −10° C. to 20° C., specifically −10° C., 0° C., or 20° C.; and the hydroxyl oxidation reaction is conducted for 0.5 h to 4 h, specifically 0.5 h, 1 h, 2 h, or 4 h.

In some embodiments, in the hydroxyl oxidation reaction, a molar ratio of the compound of Formula III to the Dess-Martin oxidant is in a range of 1:(1.0-1.5), specifically 1:(1.0-1.2) or 1:(1.2-1.5).

In some embodiments, in step 3), the reaction for constructing the terminal alkynyl group is conducted under the action of the dimethyl diazomethylphosphonate and potassium tert-butoxide (KTB) by using a water-miscible aprotic solvent with a melting point of less than −78° C., preferably tetrahydrofuran (TIF).

In some embodiments, the reaction for constructing the terminal alkynyl group is conducted at a temperature of −78° C. to −20° C., specifically −20° C.; and the reaction for constructing the terminal alkynyl group is conducted for 16 h to 48 h, specifically 16 h, 20 h, 24 h, 36 h, or 48 h.

In some embodiments, in the reaction for constructing the terminal alkynyl group, a molar ratio of the compound of Formula IV, the dimethyl diazomethylphosphonate, and the KTB is in a range of 1:(1.0-1.5):(1.0-1.5), specifically 1:1:1, 1:1.2:1.2, or 1:1.5:1.5.

In some embodiments, in step 4), the protecting group conversion reaction is conducted in the presence of a catalytic amount of sulfuric acid and under the action of acetic anhydride and acetic acid by using dichloromethane (DCM) as a solvent.

In some embodiments, the protecting group conversion reaction is conducted at a temperature of −10° C. to 35° C., specifically −10° C. to 0° C., 0° C. to 25° C., or 10° C. to 35° C.; and the protecting group conversion reaction is conducted for 4 h to 24 h, specifically 4 h, 8 h, 12 h, or 24 h.

In some embodiments, in the protecting group conversion reaction, a molar ratio of the compound of Formula V, the acetic anhydride, the acetic acid, and the sulfuric acid is in a range of 1:(5-15):(10-20):(0.01-0.10), specifically 1:5:10:0.01, 1:10:15:0.05, 1:10:20:0.0602, or 1:15:20:0.10.

In some embodiments, in step 5), the glycosylation reaction is conducted on the compound of Formula VI and 2-fluoroadenine in acetonitrile under presence of N,O-bis(trimethylsilyl)acetamide (BSA) and trimethylsilyl trifluoromethanesulfonate (TMSOTf).

In some embodiments, the glycosylation reaction is conducted at a temperature of 20° C. to 60° C., specifically 20° C., 40° C., or 60° C.; and the glycosylation reaction is conducted for 0.5 h to 12 h, specifically 0.5 h, 2 h, 6 h, or 12 h.

In some embodiments, in the glycosylation reaction, a molar ratio of the compound of Formula VI, the 2-fluoroadenine, the BSA, and the TMSOTf is in a range of 1:1:(1.2-1.5):(1.5-2.0), specifically 1:1:1.2:1.5 or 1:1:1.5:2.0.

In some embodiments, in step 6), the selective deprotection reaction is conducted under the action of a solution of ammonia in methanol.

In some embodiments, the selective deprotection reaction is conducted at a temperature of 0° C. to 40° C., specifically 0° C., 20° C., or 40° C.; and the selective deprotection reaction is conducted for 12 h to 36 h, specifically 12 h, 24 h, or 36 h.

In some embodiments, in the selective deprotection reaction, the solution of ammonia in methanol has a molar concentration of 2 mol/L to 7 mol/L, specifically 2 mol/L, 5 mol/L, or 7 mol/L, and has an amount of 5 to 20 times, specifically 5 times, 10 times, or 20 times a volume of the compound of Formula VII.

In some embodiments, in step 7), the deoxidation reaction is conducted under the action of 1,1′-thiocarbonyldiimidazole (TCDI), tributyltin hydride, and azobisisobutyronitrile (AIBN) by using toluene as a solvent.

In some embodiments, the deoxidation reaction is conducted as follows: preparing an active intermediate, and then subjecting the active intermediate to another reaction in situ to obtain the compound of Formula IX; preparing the active intermediate is conducted at a temperature of 10° C. to 30° C., specifically 10° C., 20° C., or 30° C.; preparing the active intermediate is conducted for 0.5 h to 2 h, specifically 0.5 h, 1 h, or 2 h; and the another reaction is conducted at a reflux temperature for 0.5 h to 2 h, specifically 0.5 h, 1 h, or 2 h.

In some embodiments, in the deoxidation reaction, the molar ratio of the compound of Formula VIII, the TCDI, the tributyltin hydride, and the AIBN is in a range of 1:(1.2-1.5):(2.5-4):(0.2-0.8), specifically 1(1.2-1.4):(2.5-3.5):(0.2-0.5) or 1:(1.3-1.5):(3.0-4.0):(0.5-0.8).

In some embodiments, in step 8), the debenzylation reaction is conducted under the action of boron trichloride by using DCM as a solvent.

In some embodiments, the debenzylation is conducted at a temperature of −78° C. for 0.5 h to 4 h, specifically 0.5 h, 2 h, or 4 h.

In some embodiments, in the debenzylation reaction, a molar ratio of the compound of Formula IX to the boron trichloride is in a range of 1:(1.2-7.5), specifically 1:1.2, 1:2.5, or 1:7.5.

In the present disclosure, solvents may be used in an amount well-known to those skilled in the art.

Compared with the prior art, the present disclosure has the following advantages.

1. In the present methods, the starting raw materials are easy to purchase and have low cost.

2. In the present methods, extremely dangerous reagents such as butyllithium are avoided, thereby improving the safety of the production process.

3. In the present methods, the use of protective groups is reduced, the route is shortened, and an overall yield is increased to 48.6%.

### DETAILED DESCRIPTION OF THE EMBODIMENTS

The present disclosure will be further described below in conjunction with examples, but the disclosure is not limited to the following examples. Unless otherwise specified, the process may be performed by a conventional method, and all raw materials are commercially available.

In the following examples, the compounds of Formula II to Formula IX are as follows:

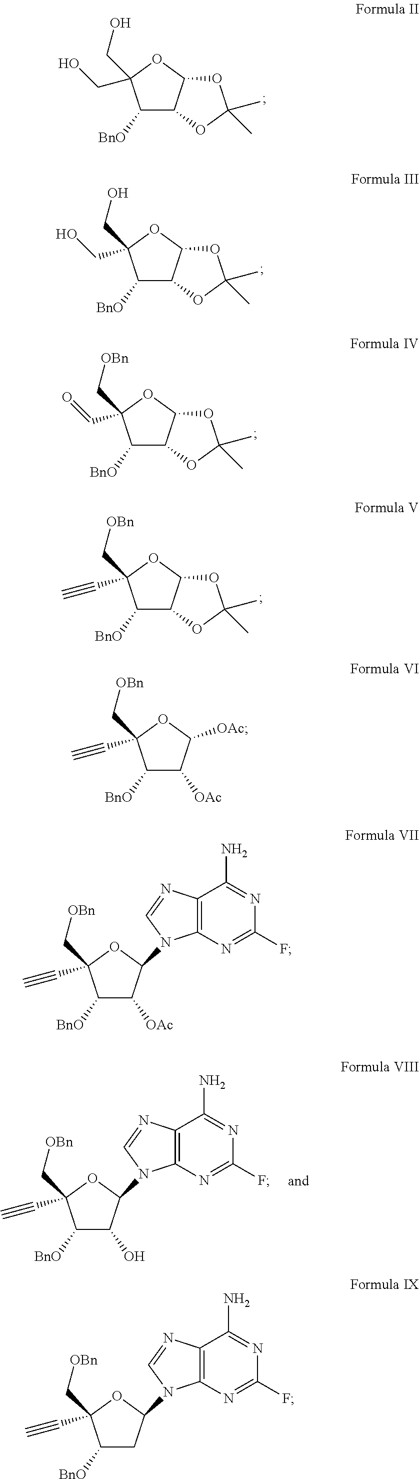

### Example 1: Synthesis of a Compound of Formula III

50.0 g (161 mmol) of a compound of Formula II was dissolved in 500 mL of DMF, and cooled to a temperature of −5° C. 7.41 g (185 mmol) of sodium hydride (with a purity of 60%) was added thereto in batches, the resulting mixture was then stirred for 1 h, and 22 mL (185 mmol) of benzyl bromide was then added dropwise thereto. After that, a resulting system was heated to a temperature of 20° C. and subjected to reaction for 16 h while stirring. After the raw materials were fully reacted, the reaction was quenched by adding water. A resulting product was extracted with DCM to obtain an organic phase, and the organic phase was then collected and recrystallized by using cyclohexane/ethyl acetate, obtaining 54.3 g of the compound of Formula III, which was a white solid with a yield of 84.2%.

### Example 2: Synthesis of a Compound of Formula IV

50.0 g (125 mmol) of the compound of Formula III was dissolved in 500 mL of DCM (previously saturated with water), and cooled to 0° C., and 63.6 g (150 mmol) of a Dess-Martin oxidant was added thereto in batches. A resulting solution was heated to 20° C. and subjected to reaction for 1 h while stirring. After the reaction was completed, a saturated sodium thiosulfate solution and a saturated sodium bicarbonate solution were added into a resulting product, and then stirred for 0.5 h to stratify; a resulting organic phase was collected, and a resulting aqueous phase was back-extracted with another DCM; organic phases were combined and washed successively with water and saturated saline solution, and then subjected to evaporation under a reduced pressure to remove solvent, obtaining 49.7 g of the compound of Formula IV as a colorless oil, ¹H NMR (400 MHz, CDCl₃) δ 9.90 (s, 1H), 7.21-7.37 (m, 10H), 5.84 (d, J=3.6 Hz, 1H), 4.65 (d, J=12.0 Hz, 2H), 4.61 (dd, J=3.6, 4.4 Hz, 1H), 4.52 (d, J=12.0 Hz, 2H), 4.37 (d, J=4.4 Hz, 1H), 3.60-3.68 (m, 2H), 1.60 (s, 3H), 1.33 (s, 3H). The above synthesis process in this example was performed by quantitative reactions, with a yield of 100%.

### Example 3: Synthesis of a Compound of Formula V

13.9 g (124 mmol) of KTB was suspended in 225 mL of THF, cooled to a temperature of −78° C.; 18.6 g (124 mmol) of dimethyl diazomethylphosphonate was added dropwise and dissolved in the 225 mL of THF solution while stirring. After that, the resulting mixture was held at the temperature of −78° C. for 0.5 h while stirring. 45.0 g (113 mmol) of the compound of Formula IV was added dropwise and dissolved in the 225 mL of THE solution; after that, a resulting system was heated to a temperature of −20° C. and subjected to reaction for 24 h. After the reaction was completed, the reaction was quenched by adding a saturated sodium bicarbonate solution; a resulting reaction solution was extracted 3 times with DCM, and resulting organic phases were combined and washed by using water and saturated saline solution in sequence, and then subjected to evaporation under a reduced pressure to remove solvent, obtaining 42.5 g of the compound of Formula V, which was a colorless oil with a yield of 95.4%.

### Example 4: Synthesis of a Compound of Formula VI

40.0 g (101 mmol) of the compound of Formula V was dissolved in 400 mL of DCM, and cooled to a temperature of 0° C.; 116 mL (2.03 mol) of acetic acid, 95 mL (1.01 mol) of acetic anhydride, and 0.3 mL (6.08 mmol) of sulfuric acid was successively added into a resulting solution. After that, a resulting system was held at 0° C. and subjected to reaction for 12 h while stirring; after the reaction was completed, a saturated sodium bicarbonate solution was added into a resulting reaction solution to make the resulting reaction solution to be neutrality. A resulting neutral liquid was subjected to liquid separation, and a resulting organic phase was collected, and a resulting aqueous phase was back-extracted with another DCM, organic phases were combined and washed with saturated sodium bicarbonate, and then subjected to evaporation under a reduced pressure to remove solvent, obtaining 39.8 g of the compound of Formula VI, which was a colorless oil with a yield of 89.5%.

### Example 5: Synthesis of a Compound of Formula VII

12.2 g (79.8 mmol) of 2-fluoroadenine was suspended in 350 mL of acetonitrile, heated to a temperature of 40° C., and 23 mL (95.8 mmol) of BSA was added thereto; after a resulting mixed solution was clear, 35.0 g (79.8 mmol) of the compound of Formula VI and 22 mL (120 mmol) of TMSOTf were added thereto in sequence. A resulting system was held at the temperature of 40° C., and subjected to reaction while stirring for 6 h. After the reaction was completed, a resulting reaction solution was diluted by adding ethyl acetate, and ice water was then added thereto; a resulting liquid were subjected to liquid separation, and a resulting organic phase was collected and washed with a saturated saline solution, and then evaporated to dryness under a reduced pressure, obtaining 38.6 g of the compound of Formula VII, which was a white-like powder with a yield of 91.0%.

### Example 6: Synthesis of a Compound of Formula VIII

35.0 g (65.8 mmol) of the compound of Formula VII was suspended in 350 mL of a solution of ammonia in methanol (5 mol/L), and a resulting system was subjected to reaction at 0° C. for 24 h while stirring. After the reaction was completed, a resulting reaction solution was evaporated to dryness under a reduced pressure, and then recrystallized by using n-hexane/ethyl acetate, obtaining 30.7 g of the compound of Formula VIII, which was a white-like powder with a yield of 95.2%.

### Example 7: Synthesis of a Compound of Formula IX

30 g (61.3 mmol) of the compound of Formula VIII was dissolved in 500 mL of toluene, and 13.1 g (73.5 mmol) of TCDI was added into a resulting solution. A resulting system was subjected to reaction while stirring at 20° C. for 1 h; after that, 49 mL (184 mmol) of tributyltin hydride and 4.5 mL (30.6 mmol) of AIBN were added thereto. A resulting mixed solution was heated until a reflux was formed and subjected to another reaction for 1 h. After the another reaction was completed, a resulting product was cooled, and evaporated to remove solvent under a reduced pressure, and then recrystallized by using n-hexane/ethyl acetate, obtaining 27.8 g of the compound of Formula IX, with a yield of 95.8%.

### Example 8: Synthesis of Islatravir

25.0 g (52.8 mmol) of the compound of Formula IX was dissolved in 125 mL of DCM, and cooled to a temperature of −78° C., and a pre-prepared solution of boron trichloride in DCM (prepared by dissolving 15.5 g, 132 mmol of boron trichloride in 125 mL of another DCM) was added dropwise. After that, a resulting system was subjected to reaction for 2 h at the temperature; after the reaction was completed, the reaction was quenched by adding a 1:1 (v/v) DCM/methanol mixture. A resulting product was evaporated to dryness under a reduced pressure, and a resulting residue was recrystallized by using ethanol, obtaining 12.6 g of the islatravir, which was a white-like powder with a yield of 81.4%, ¹H NMR (400 MHz, DMSO-d₆) δ 8.30 (s, 1H), 7.85 (br s, 2H), 6.24 (dd, J=7.3, 5.0 Hz, 1H), 5.58 (d, J=5.4 Hz, 1H), 5.30 (t, J=6.2 Hz, 1H), 4.54-4.59 (m, 1H), 3.65 (dd, J=11.9, 5.6 Hz, 1H), 3.55 (dd, J=11.9, 6.7 Hz, 1H), 3.51 (s, 1H), 2.67-2.73 (m, 1H), 2.39-2.46 (m, 1H).

Although the present disclosure is described in detail in conjunction with the foregoing examples, they are only a part of, not all of, the embodiments of the present disclosure. Other embodiments can be obtained based on these examples without creative efforts, and all of these embodiments shall fall within the scope of the present disclosure.

1. A method for preparing islatravir, comprising the following steps:
1) subjecting a compound of Formula II as a raw material to selective hydroxyl protection reaction to obtain a compound of Formula III, 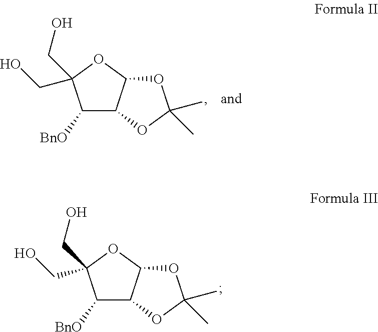
2) subjecting the compound of Formula III to hydroxyl oxidation reaction to obtain a compound of Formula IV, 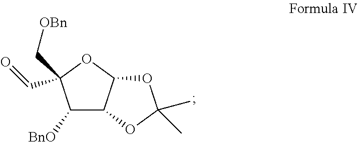 [SMILES: CC1(C)O[C@H]2O[C@](C=O)(COCc3ccccc3)[C@@H](OCc3ccccc3)[C@H]2O1]
3) subjecting the compound of Formula IV and dimethyl diazomethylphosphonate to reaction under an alkaline condition to obtain a compound of Formula V, 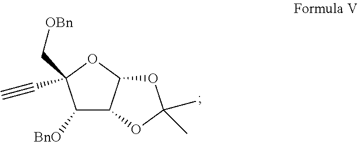 [SMILES: C#C[C@]1(COCc2ccccc2)O[C@@H]2OC(C)(C)O[C@@H]2[C@@H]1OCc1ccccc1]
4) subjecting the compound of Formula V to protecting group conversion reaction, such that protection by one propylidene group is converted to protection by two acetyl groups to obtain a compound of Formula VI, 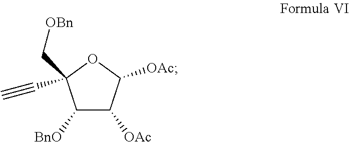 [SMILES: C#C[C@]1(COCc2ccccc2)O[C@H](OC(C)=O)[C@H](OC(C)=O)[C@@H]1OCc1ccccc1]
5) subjecting the compound of Formula VI to glycosylation reaction, such that a glycosidic bond is constructed to obtain a compound of Formula VII, 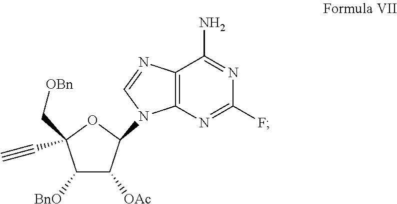 [SMILES: C#C[C@]1(COCc2ccccc2)O[C@@H](n2cnc3c(N)nc(F)nc32)[C@H](OC(C)=O)[C@@H]1OCc1ccccc1]
6) subjecting the compound of Formula VII to selective deprotection reaction to remove the acetyl groups to obtain a compound of Formula VIII, 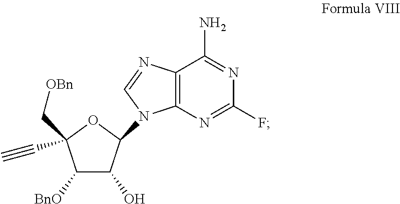 [SMILES: C#C[C@]1(COCc2ccccc2)O[C@@H](n2cnc3c(N)nc(F)nc32)[C@H](O)[C@@H]1OCc1ccccc1]
7) subjecting the compound of Formula VIII to deoxidation reaction to remove a hydroxyl group at a 2′-position to obtain a compound of Formula IX, 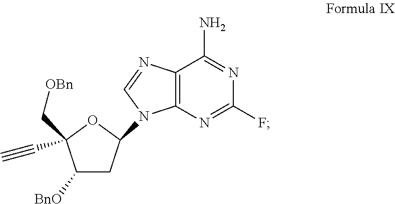 [SMILES: C#C[C@]1(COCc2ccccc2)O[C@@H](n2cnc3c(N)nc(F)nc32)C[C@@H]1OCc1ccccc1]
and
8) subjecting the compound of Formula IX to debenzylation reaction to remove two benzyl groups to obtain the islatravir.

2. The method of claim 1, wherein in step 1), the selective hydroxyl protection reaction is conducted under the action of sodium hydride and benzyl bromide by using N,N-dimethylformamide (DMF) as a solvent;
the selective hydroxyl protection reaction is conducted at a temperature of −10° C. to 40° C. for 6 hours to 24 hours; and
a molar ratio of the compound of Formula II, the sodium hydride, and the benzyl bromide is in a range of 1:(0.8-1.2):(0.8-1.2).

3. The method of claim 2, wherein the molar ratio of the compound of Formula II, the sodium hydride, and the benzyl bromide is in a range of 1:(0.8-1.0):(0.8-1.0), or 1:(1.0-1.2):(1.0-1.2).

4. The method of claim 1, wherein in step 2), the hydroxyl oxidation reaction is conducted under the action of a Dess-Martin oxidant by using dichloromethane (DCM) as a further solvent;
the hydroxyl oxidation reaction is conducted at a temperature of −10° C. to 20° C. for 0.5 hours to 4 hours; and
a molar ratio of the compound of Formula III to the Dess-Martin oxidant is in a range of 1:(1.0-1.5).

5. The method of claim 4, wherein the molar ratio of the compound of Formula III to the Dess-Martin oxidant is in a range of 1:(1.0-1.2), or 1:(1.2-1.5).

6. The method of claim 1, wherein in step 3), the reaction is conducted under the action of the dimethyl diazomethylphosphonate and potassium tert-butoxide (KTB) by using a water-miscible aprotic solvent with a melting point of less than −78° C.;
the reaction is conducted at a temperature of −78° C. to −20° C. for 16 hours to 48 hours; and
a molar ratio of the compound of Formula IV, the dimethyl diazomethylphosphonate, and the KTB is in a range of 1:(1.0-1.5):(1.0-1.5).

7. The method of claim 6, wherein the water-miscible aprotic solvent with a melting point of less than −78° C. is tetrahydrofuran (THF).

8. The method of claim 1, wherein in step 4), the protecting group conversion reaction is conducted in the presence of a catalytic amount of sulfuric acid and under the action of acetic anhydride and acetic acid by using dichloromethane (DCM) as a solvent;
the protecting group conversion reaction is conducted at a temperature of −10° C. to 35° C. for 4 hours to 24 hours; and
a molar ratio of the compound of Formula V, the acetic anhydride, the acetic acid, and the sulfuric acid is in a range of 1:(5-15):(10-20):(0.01-0.10).

9. The method of claim 1, wherein in step 5), the glycosylation reaction is conducted on the compound of Formula VI and 2-fluoroadenine in acetonitrile in the presence of N,O-bis(trimethylsilyl)acetamide (BSA) and trimethylsilyl trifluoromethanesulfonate (TMSOTf);
the glycosylation reaction is conducted at a temperature of 20° C. to 60° C. for 0.5 hours to 12 hours; and
a molar ratio of the compound of Formula VI, the 2-fluoroadenine, the BSA, and the TMSOTf is in a range of 1:1:(1.2-1.5):(1.5-2.0).

10. The method of claim 1, wherein in step 6), the selective deprotection reaction is conducted under the action of a solution of ammonia in methanol;
the selective deprotection reaction is conducted at a temperature of 0° C. to 40° C. for 12 hours to 36 hours; and
in the selective deprotection reaction, the solution of ammonia in methanol has a molar concentration of 2 mol/L to 7 mol/L, and is used in an amount of 5 to 20 times volume of the compound of Formula VII.

11. The method of claim 1, wherein in step 7), the deoxidation reaction is conducted under the action of 1,1′-thiocarbonyldiimidazole (TCDI), tributyltin hydride, and azobisisobutyronitrile (AIBN) by using toluene as a solvent; and
a molar ratio of the compound of Formula VIII, the TCDI, the tributyltin hydride, and the AIBN is in a range of 1:(1.2-1.5):(2.5-4):(0.2-0.8).

12. The method of claim 11, wherein the molar ratio of the compound of Formula VIII, the TCDI, the tributyltin hydride, and the AIBN is in a range of 1:(1.2-1.4):(2.5-3.5):(0.2-0.5), or 1:(1.3-1.5):(3.0-4.0):(0.5-0.8).

13. The method of claim 11, wherein the deoxidation reaction is conducted by preparing an active intermediate, and then subjecting the active intermediate to another reaction in situ to obtain the compound of Formula IX;
wherein preparing the active intermediate is conducted at a temperature of 10° C. to 30° C. for 0.5 hours to 2 hours; and
the another reaction is conducted at a reflux temperature for 0.5 hours to 2 hours.

14. The method of claim 1, wherein in step 8), the debenzylation reaction is conducted under the action of boron trichloride by using DCM as a solvent;
the debenzylation reaction is conducted at a temperature of −78° C. for 0.5 hours to 4 hours;
and a molar ratio of the compound of Formula IX to the boron trichloride is in a range of 1:(1.2-7.5).

15. The method of claim 2, wherein in step 2), the hydroxyl oxidation reaction is conducted under the action of a Dess-Martin oxidant by using dichloromethane (DCM) as a further solvent;
the hydroxyl oxidation reaction is conducted at a temperature of −10° C. to 20° C. for 0.5 hours to 4 hours; and
a molar ratio of the compound of Formula III to the Dess-Martin oxidant is in a range of 1:(1.0-1.5)
